(19) United States
(12) Patent Application Publication
(10) Pub. No.: US 2026/0263285 A1
(43) Pub. Date: Sep. 10, 2026

(54) NEGATIVE PRESSURE WOUND THERAPY APPARATUSES AND SYSTEMS

(71) Applicant: Solventum Intellectual Properties Company, Maplewood, MN (US)

(72) Inventors: Christoph SCHUELL, Neuss (DE); Hironobu ISHIWATARI, Neuss (DE); James A. LUCKEMEYER, Saint Paul, MN (US); Dennis J. POHL, Saint Paul, MN (US); David R. HOLM, Saint Paul, MN (US); Kiu-Yuen TSE, Saint Paul, MN (US); Simone JURJEVIC, Neuss (DE); Daniel W. DEKRUIF, Neuss (DE)

(21) Appl. No.: 18/871,765

(22) PCT Filed: May 31, 2023
(86) PCT No.: PCT/IB2023/055600

§ 371 Date: Dec. 4, 2024

Related U.S. Application Data
(60) Provisional application No. 63/349,375, filed on Jun. 6, 2022.

(51) Int. Cl. A61F 13/05; A61F 13/00; A61F 13/02; A61F 13/0246; A61M 1/00

(52) U.S. Cl. CPC A61F 13/05; A61F 13/0253; A61F 13/0256; A61F 13/0266; A61M 1/915; A61F 2013/00612; A61F 2013/0074

(57) ABSTRACT

Apparatuses and systems for use in a negative pressure wound therapy environment. A dressing configured to be positioned at a tissue site comprises a first film layer, a manifold, and a cover layer. The first film layer comprises a treatment region with a first plurality of perforations, a sealing region around the treatment region with a second plurality of perforations, and a sealing adhesive covering the sealing region on a first side of the first film layer configured to face the tissue site. The manifold is disposed adjacent to a second side of the first film layer where the second side is opposite the first side. The cover layer is disposed over the manifold and coupled to the second side of the first film layer around the manifold. The cover layer comprises a second film layer and a cover adhesive disposed adjacent to the second plurality of perforations.

### CROSS-REFERENCE TO RELATED APPLICATIONS

This application is a U.S. National Stage Entry of PCT International Application No. PCT/IB2023/055600, filed on May 31, 2023, which claims the benefit of priority to U.S. Provisional Application No. 63/349,375, filed on Jun. 6, 2022, which is incorporated herein by reference in its entirety.

### TECHNICAL FIELD

The invention set forth in the appended claims relates generally to tissue treatment systems and more particularly, but without limitation, to dressings for tissue treatment with negative pressure and methods of using the dressings for tissue treatment with negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a dressing for treating tissue can include a first film layer, a manifold, and a cover layer. The first film layer can include a treatment region and a sealing region around the treatment region. The first film layer can further include a first plurality of perforations formed through the treatment region and a second plurality of perforations formed through the sealing region. The second plurality of perforations can be larger than the first plurality of perforations. The first film layer can further include a sealing adhesive covering the sealing region on a first side of the first film layer configured to face the tissue site. The manifold can be disposed adjacent to a second side of the first film layer. The second side of the first film layer can be opposite the first side of the first film layer. The cover layer can include a second film layer and a cover adhesive. The cover layer can be disposed over the manifold and can be coupled to the second side of the first film layer around the manifold. The cover adhesive can be disposed adjacent to the second plurality of perforations.

In some example embodiments, the first film layer can include a polyurethane film having a thickness of between about 20 microns and about 35 microns.

In some example embodiments, the sealing adhesive can include a silicone adhesive having a coating weight of between about 100 grams per square meter and about 250 grams per square meter.

In some example embodiments, the manifold can include an open-cell foam having a thickness of between about 5 millimeters and about 10 millimeters.

In some example embodiments, the second film layer can include a polyurethane film having a thickness of between about 20 microns and about 35 microns.

In some example embodiments, the cover adhesive can include an acrylic adhesive having a coating weight of between about 25 grams per square meter and about 65 grams per square meter.

In some example embodiments, the cover adhesive can be configured to extend through the second plurality of perforations from the second side of the first film layer to the first side of the first film layer.

In some example embodiments, the treatment region can be configured to contact the tissue site and the sealing region can be configured to contact epidermis around the tissue site.

In some example embodiments, the treatment region can be free of adhesive such that the first side of the first film layer in the treatment region is configured to be positioned in direct contact with the tissue site.

In some example embodiments, the first plurality of perforations can include slits arranged in a pattern.

In some example embodiments, the second plurality of perforations can include circular openings arranged in a pattern.

In some example embodiments, each of the first plurality of perforations can include a first open area and each of the second plurality of perforations can include a second open area that is larger than the first open area.

In some example embodiments, the first plurality of perforations can include a different shape than the second plurality of perforations.

In some example embodiments, the second plurality of perforations can be larger than the first plurality of perforations in at least one dimension.

In some example embodiments, the first plurality of perforations can be configured to increase in size when the tissue site is exposed to a pressure. The second plurality of perforations do not change in size when exposed to the pressure. The second plurality of perforations can be larger in size than the first plurality of perforations when the tissue site is exposed to the pressure and when the tissue site is not exposed to the pressure.

In some example embodiments, the dressing can further include a carrier layer releasably coupled to a side of the cover layer opposite the cover adhesive. The carrier layer can include a coated paper or a polymeric film.

Also described herein is an example system for treating a tissue site with negative-pressure therapy. The system can include a dressing that can include a first film layer, a manifold, and a cover layer. The first film layer can include a treatment region and a sealing region around the treatment region. The first film layer can further include a first plurality of perforations formed through the treatment region and a second plurality of perforations formed through the sealing region. The second plurality of perforations can be larger than the first plurality of perforations. The first film layer can further include a sealing adhesive covering the sealing region on a first side of the first film layer configured to face the tissue site. The manifold can be disposed adjacent to a second side of the first film layer. The second side of the first film layer can be opposite the first side of the first film layer. The cover layer can include a second film layer and a cover adhesive. The cover layer can be disposed over the manifold and can be coupled to the second side of the first film layer around the manifold. The cover adhesive can be disposed adjacent to the second plurality of perforations. The system can also include a negative-pressure source configured to be fluidly coupled to the tissue site through the cover.

Also described herein is another example system for treating a tissue site with negative-pressure therapy. The system can include a dressing configured to be positioned adjacent to the tissue site. The dressing can include a first film layer, a manifold, and a second film layer. The first film layer can include a treatment region and a sealing region around the treatment region. A plurality of slits can be formed through the treatment region and a plurality of holes can be formed through the sealing region. The plurality of holes can have a larger open area than the plurality of slits. The first film layer can further include a sealing adhesive covering the sealing region on a side of the first film layer configured to face the tissue site. The second film layer can include an aperture. The second film layer can be coated with a pressure-sensitive adhesive on a side of the second film layer configured to face the tissue site. The carrier layer, the first film layer, the manifold, and the second film layer can be assembled in a stacked relationship with the first film layer and the second film layer enclosing the manifold. The manifold can be aligned with the treatment region. The pressure-sensitive adhesive and the sealing region can be configured to face the tissue site and at least some of the pressure-sensitive adhesive can be exposed through the plurality of holes. The carrier layer can be laminated to the second film layer on a side of the second film layer opposite the pressure-sensitive adhesive. A negative-pressure source can be configured to be fluidly coupled to the tissue site through the aperture, the manifold, and the plurality of slits.

In some example embodiments, the sealing adhesive is not disposed on the treatment region.

In some example embodiments, the carrier layer can include a coated paper or a polymeric film. The carrier layer can be releasably coupled to the second film layer and can be configured to be removed from the second film layer after the dressing is positioned adjacent to the tissue site.

In some example embodiments, the sealing adhesive can be applied in a pattern to the first film layer. The sealing adhesive can be applied in a pattern to the first film layer such that the treatment region is free of the sealing adhesive. The sealing adhesive can be applied in a pattern to the first film layer such that the sealing region includes the sealing adhesive, but the treatment region is free of the sealing adhesive.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide tissue treatment in accordance with this specification;

FIG. 2 is an exploded view of an example of a dressing of the therapy system of FIG. 1;

FIG. 3 is an assembly view of the dressing of FIG. 2;

FIG. 4A is a top view of an example layer of the dressing of FIG. 2 illustrating additional details that may be associated with some embodiments;

FIG. 4B is a bottom view of the example layer of FIG. 4 illustrating additional details that may be associated with some embodiments;

FIG. 5 is a cut-away view of an illustrative embodiment of the therapy system of FIG. 1 depicting an illustrative example embodiment of a dressing interface and a dressing deployed at a tissue site;

FIG. 5A is a detail view of the dressing of FIG. 5 taken at reference 5A, depicted in FIG. 5;

FIG. 5B is another detail view of the dressing of FIG. 5A;

FIG. 6A is an exploded view of another embodiment of a dressing of the therapy system of FIG. 1; and

FIG. 6B is an assembly view of the dressing of FIG. 6A.

### DESCRIPTION OF EXAMPLE EMBODIMENTS

The following description of example embodiments provides information that enables a person skilled in the art to make and use the subject matter set forth in the appended claims, and may omit certain details already well-known in the art. The following detailed description is, therefore, to be taken as illustrative and not limiting.

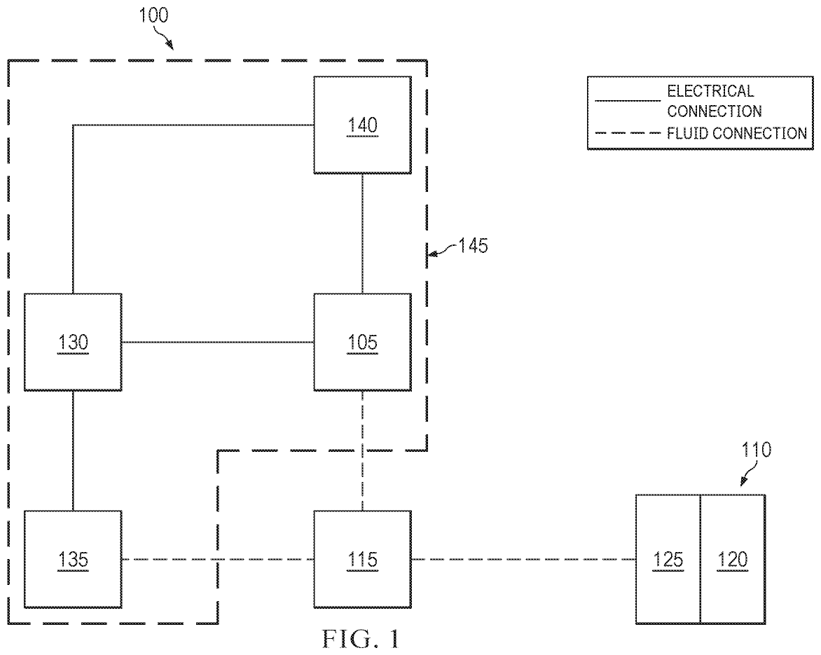

FIG. 1 is a block diagram of an example embodiment of a therapy system 100 that can provide negative-pressure therapy to a tissue site in accordance with this specification.

The term “tissue site” in this context broadly refers to a wound, defect, or other treatment target located on or within tissue, including but not limited to, a surface wound, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from 3M Company.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from a tissue site and drawing the fluid toward the source. In some embodiments, the fluid path may be reversed, or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from 3M Company.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from 3M Company. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and caprolactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, polyurethane (PU) drape, commercially available from Avery Dennison Corporation, Pasadena, California; polyether block polyamide copolymer (PEBAX), for example, from Arkema S.A., Colombes, France; and INSPIRE® 2301 and INSPIRE® 2327 polyurethane films, commercially available from Exopack Advanced Coatings, Wrexham, United Kingdom. In some embodiments, the cover 125 may comprise INSPIRE® 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example.

In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as limiting.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

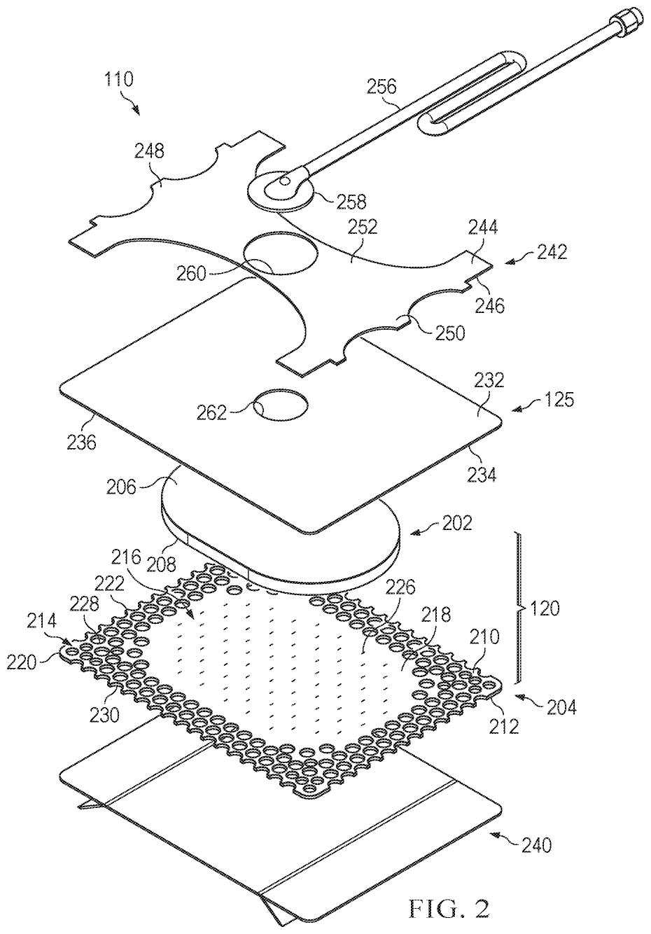

FIG. 2 is an exploded view of an example embodiment of the dressing 110 of FIG. 1, illustrating additional details that may be associated with some embodiments in which the tissue interface 120 includes more than one layer. In some embodiments, the tissue interface 120 may include a manifold 202 and a first film layer 204. The manifold 202 may include a first surface 206 and a second surface 208 opposite the first surface 206. The first film layer 204 may include a first surface 210 and a second surface 212 opposite the first surface 210. The first surface 210 of the first film layer 204 may couple to the second surface 208 of the manifold 202 such that the manifold 202 is stacked on top of the first film layer 204 when the dressing 110 is assembled.

The manifold 202 may comprise or consist essentially of a manifold or manifold layer as previously described with reference to FIG. 1, which provides a means for collecting or distributing fluid across the tissue interface 120 under pressure. The manifold 202 may be ovular or a stadium shaped manifold in some embodiments. In some embodiments, the manifold 202 may be GRANUFOAM™ or an open-cell foam that may be between about 5 millimeters and about 10 millimeters thick. The manifold 202 may be adapted to receive negative pressure from a source, such as the negative-pressure source 105, and distribute negative pressure through multiple apertures across the tissue interface 120, which may have the effect of collecting fluid from a tissue site and drawing the fluid toward the source. In some embodiments, the fluid path may be reversed, or a secondary fluid path may be provided to facilitate delivering fluid, such as from a source of instillation solution, across the tissue interface 120.

The first film layer 204 may comprise or consist essentially of a means for controlling or managing fluid flow. The first film layer 204 may include a periphery 214 that may surround an interior portion 216. The interior portion 216 may be symmetrically and centrally disposed in the first film layer 204. In some embodiments, the periphery 214 may be a sealing region of the first film layer 204 and the interior portion 216 may be a treatment region of the first film layer 204. In some embodiments, the interior portion 216 may correspond to a surface area of the manifold 202. In some embodiments, a border 218 may divide or separate the interior portion 216 from the periphery 214 of the first film layer 204. The first film layer 204 may further include corners 220 and edges 222 that may be part of the periphery 214.

The first film layer 204 may further include a first plurality of perforations 226 formed through the interior portion 216 and a second plurality of perforations 228 formed through the periphery 214. The first plurality of perforations 226 may be fluid restrictions that may be bi-directional and pressure responsive. The first plurality of perforations 226 can generally comprise or consist essentially of an elastic passage that can expand in response to a pressure gradient. In some embodiments, the first plurality of perforations 226 may be formed by removing material from the first film layer 204. For example, the first plurality of perforations 226 may be formed by cutting through the first film layer 204, which may also deform the edges of the first plurality of perforations 226 in some embodiments. In the absence of a pressure gradient across the first plurality of perforations 226, the passages may be sufficiently small to form a seal or flow restriction, which can substantially reduce or prevent liquid flow. Additionally or alternatively, one or more of the first plurality of perforations 226 may be an elastomeric valve that is normally closed when unstrained, or not subject to negative pressure, to substantially prevent liquid flow, and can open in response to a pressure gradient.

In some embodiments, the first plurality of perforations 226 may comprise or consist essentially of one or more slits or slots or combinations of slits or slots in the first film layer 204. In some examples, the first plurality of perforations 226 may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications. A tolerance of about 0.1 millimeters may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

In some embodiments, the first plurality of perforations 226 may be slits or slots arranged in a pattern. For example, the slits or slots may be arranged in a chevron pattern, in a grid of parallel rows and columns, or in another pattern. Alternatively, the first plurality of perforations 226 may be arranged randomly throughout the interior portion 216 of the first film layer 204.

The second plurality of perforations 228 may be disposed through the periphery 214 of the first film layer 204. The second plurality of perforations 228 may be apertures or holes that may be formed by cutting or by application of local RF or ultrasonic energy, for example, or by other suitable techniques for forming an opening. The second plurality of perforations 228 may have a uniform distribution pattern or may be randomly distributed on the periphery 214 of the first film layer 204. The second plurality of perforations 228 may have many shapes, including circles, squares, stars, ovals, polygons, slits, complex curves, rectilinear shapes, triangles, for example, or may have some combination of such shapes.

Each of the second plurality of perforations 228 may have uniform or similar geometric properties. For example, in some embodiments, each of the second plurality of perforations 228 may be circular apertures, having substantially the same diameter. In some embodiments, the diameter of each of the second plurality of perforations 228 may be between about 1 millimeter to about 50 millimeters. In other embodiments, the diameter of each of the second plurality of perforations 228 may be between about 1 millimeter to about 20 millimeters.

In other embodiments, geometric properties of the second plurality of perforations 228 may vary. For example, the diameter of the second plurality of perforations 228 may vary depending on the position of the second plurality of perforations 228. In some embodiments, the second plurality of perforations 228 disposed in the corners 220 may have a diameter between about 7.75 millimeters to about 8.75 and the remainder of the second plurality of perforations 228 may have a diameter between about 9.8 millimeters to about 10.2 millimeters.

At least one of the second plurality of perforations 228 may be positioned at the edges 222 of the periphery 214 and may have an interior cut open or exposed at the edges 222 that is in fluid communication in a lateral direction with the edges 222. The lateral direction may refer to a direction toward the edges 222 and in the same plane as the first film layer 204. The second plurality of perforations 228 positioned proximate to or at the edges 222 may be spaced substantially equidistant around the periphery 214. Alternatively, the spacing of the second plurality of perforations 228 proximate to or at the edges 222 may be irregular.

In some embodiments, each of the first plurality of perforations 226 may have a first open area and each of the second plurality of perforations 228 may have a second open area. Each second open area may be larger than each first open area. Similarly, each of the second plurality of perforations 228 may be larger than each of the first plurality of perforations 226 in at least one dimension. As described above, each of the first plurality of perforations 226 may be pressure responsive and thus, each of the first plurality of perforations 226 may be configured to increase in size when the tissue site is exposed to a pressure such as a negative pressure from the negative-pressure source 105. In contrast, the second plurality of perforations 228 may not change in size when exposed to the pressure. Further, the second plurality of perforations 228 may be larger in size than the first plurality of perforations 226 when the tissue site is exposed to the pressure and when the tissue site is not exposed to the pressure.

In some embodiments, the first film layer 204 may further include a sealing adhesive 230. The sealing adhesive 230 may be disposed on the second surface 212 of the first film layer 204 such that the sealing adhesive 230 will face a tissue site and contact a periwound region of the tissue site when the dressing 110 is placed at the tissue site. The sealing adhesive 230 may be disposed on the periphery 214 and may not be disposed on the interior portion 216 of the first film layer 204. The sealing adhesive 230 may be applied in a pattern onto the first film layer 204 such that the interior portion 216 is free from the sealing adhesive 230. In some embodiments, the sealing adhesive 230 can be or can include a coating of a silicone material. Suitable silicone adhesives are described, for example, in US Patent Publication 2011/0212325 and are incorporated herein for reference. In some embodiments, silicone oils such as OHX4070 from Dow and AK1,000,000 from Wacker may be included in the sealing adhesive 230. In some embodiments, the sealing adhesive 230 may have a coating weight of between about 100 grams per square meter and about 250 grams per square meter.

The first film layer 204 may comprise or consist essentially of a film that may provide a fluid seal with the tissue site and may have a substantially flat surface. The first film layer 204 may be a polyurethane film such as Pellethane® 5863-86A (Lubrizol) in some embodiments. The first film layer 204 may be between about 20 microns to about 35 microns thick in some embodiments. In some embodiments, the first film layer 204 may further include an adhesive on the first surface 210 of the first film layer 204. The adhesive on the first surface 210 may adhere the first film layer 204 to the cover 125 and/or the manifold 202. The adhesive on the first surface 210 of the first film layer 204 may include any of the materials described above for the attachment device with reference to FIG. 1.

The dressing 110 may further include a second film layer such as the cover 125. The cover 125 may include a first surface 232 and a second surface 234 opposite the first surface 232. The second surface 234 of the cover 125 may be configured to couple to the first surface 206 of the manifold 202 of the tissue interface 120. The cover 125 may comprise or consist essentially of a polyurethane film such as Pellethane® 5863-86A (Lubrizol). In some embodiments, the cover 125 may have a thickness of between about 20 microns and about 35 microns. The dressing 110 may further include an attachment device such as the attachment device described above with reference to FIG. 1 or a cover adhesive or an adhesive 236. The adhesive 236 may be, for example, a medically-acceptable, pressure-sensitive adhesive that extends about a periphery, a portion, or the entirety of the second surface 234 of the cover 125. In some embodiments, for example, the adhesive 236 may be an acrylic adhesive having a coating weight between about 25 g.s.m. and about 65 g.s.m. Thicker adhesives, or combinations of adhesives, may be applied in some embodiments to improve the seal and reduce leaks. The adhesive 236 may be a layer having substantially the same shape as the periphery 214 of the first film layer 204. In some embodiments, the adhesive 236 may be continuous or discontinuous. Discontinuities in the adhesive 236 may be provided by apertures or holes (not shown) in the adhesive 236. The apertures or holes in the adhesive 236 may be formed after application of the adhesive 236 or by applying the adhesive 236 in patterns on the second surface 234 of the cover 125. Apertures or holes in the adhesive 236 may also be sized to enhance the MVTR of the dressing 110 in some example embodiments.

As illustrated in the example of FIG. 2, in some embodiments, a release liner 240 may be attached to or positioned adjacent to the first film layer 204 to protect the adhesive 236 prior to use. The release liner 240 may also provide stiffness to assist with, for example, deployment of the dressing 110. The release liner 240 may be, for example, a casting paper, a film, or polyethylene. Further, in some embodiments, the release liner 240 may be a polyester material such as polyethylene terephthalate (PET), or similar polar semi-crystalline polymer. The use of a polar semi-crystalline polymer for the release liner 240 may substantially preclude wrinkling or other deformation of the dressing 110. For example, the polar semi-crystalline polymer may be highly orientated and resistant to softening, swelling, or other deformation that may occur when brought into contact with components of the dressing 110, or when subjected to temperature or environmental variations, or sterilization. In some embodiments, the release liner 240 may have a surface texture that may be imprinted on an adjacent layer, such as the first film layer 204. In some embodiments, the release liner 240 may be Scotchpak™ Fluoropolymer Coated Release Liner (3M). Further, a release agent may be disposed on a side of the release liner 240 that is configured to contact the first film layer 204. For example, the release agent may be a silicone coating and may have a release factor suitable to facilitate removal of the release liner 240 by hand and without damaging or deforming the dressing 110. In some embodiments, the release agent may be a fluorocarbon or a fluorosilicone, for example. In other embodiments, the release liner 240 may be uncoated or otherwise used without a release agent.

In some embodiments, the dressing 110 may further include a carrier 242. The carrier 242 may include a first surface 244 and a second surface 246 opposite the first surface 244. The second surface 246 of the carrier 242 may be configured to couple to the first surface 232 of the cover 125. In some embodiments, the carrier 242 may be releasably coupled to the first surface 232 of the cover 125. In some embodiments, the carrier 242 may be laminated to the first surface 232 of the cover 125. The carrier 242 may enable stable handling after the release liner 240 is removed from the dressing 110. In some embodiments, the carrier 242 may have a first elongate portion 248 and a second elongate portion 250 connected by a connecting portion 252. The carrier 242 may have another shape or size in some embodiments but may still provide structure for the dressing 110 after the release liner 240 is removed. The carrier 242 may be comprised of coated paper or film in some embodiments. Examples of carrier material may include polyethylene/vinyl acetate copolymer coated Kraft paper available from Loparex.

In some embodiments, the dressing 110 may further include optional handling bars (not pictured). The optional handling bars may provide a place for a health care practitioner or a user to hold the dressing 110 while applying it to a tissue site. In some embodiments, the optional handling bars may be coupled to the adhesive 236 of the cover 125. In other embodiments the optional handling bars may be located at a different position on the dressing 110.

FIG. 2 also illustrates an example embodiment of a fluid conductor 256 and a dressing interface 258. The fluid conductor 256 may be a flexible tube, which may be fluidly coupled on one end to the dressing interface 258. The dressing interface 258 may be an elbow connector, which can be placed through an aperture 260 in the carrier 242 that may surround an aperture 262 in the cover 125. The aperture 262 may provide a fluid path between the fluid conductor 256 and the tissue interface 120 of the dressing 110.

In use, the release liner 240 (if included) may be removed to expose the first film layer 204, which may be placed within, over, on, or otherwise proximate to a tissue site, particularly a surface tissue site and adjacent epidermis. The first film layer 204 may be interposed between the manifold 202 and the tissue site, which can substantially reduce or eliminate adverse interaction with the manifold 202. For example, the first film layer 204 may be placed over a surface wound (including edges of the wound) and undamaged epidermis to prevent direct contact with the manifold 202. Treatment of a surface wound or placement of the dressing 110 on a surface wound includes placing the dressing 110 immediately adjacent to the surface of the body or extending over at least a portion of the surface of the body. Treatment of a surface wound does not include placing the dressing 110 wholly within the body or wholly under the surface of the body, such as placing a dressing within an abdominal cavity. In some applications, the interior portion 216 of the first film layer 204 may be positioned adjacent to, proximate to, or covering a tissue site. The periphery 214 of the first film layer 204 may be positioned adjacent to or proximate to tissue around or surrounding the tissue site. The first film layer 204 may be sufficiently tacky to hold the dressing 110 in position, while also allowing the dressing 110 to be removed or re-positioned without trauma to the tissue site.

Removing the release liner 240 can also expose the adhesive 236 of the cover 125 to a tissue site. For example, the cover 125 may be attached to epidermis peripheral to a tissue site, around the manifold 202. The adhesive 236 may be in fluid communication with an attachment surface through the second plurality of perforations 228 in at least the periphery 214 of the first film layer 204 in some embodiments. The adhesive 236 may also be in fluid communication with the edges 222 through the second plurality of perforations 228 exposed at the edges 222.

Once the dressing 110 is in the desired position, the adhesive 236 may be pressed through the second plurality of perforations 228 to bond the dressing 110 to an attachment surface. The second plurality of perforations 228 at the edges 222 may permit the adhesive 236 to flow around the edges 222 for enhancing the adhesion of the edges 222 to an attachment surface.

In some embodiments, the second plurality of perforations 228 in the first film layer 204 may be sized to control the amount of the adhesive 236 in fluid communication with an attachment surface though the second plurality of perforations 228. For a given geometry of the corners 220, the relative sizes of the second plurality of perforations 228 may be configured to maximize the surface area of the adhesive 236 exposed and in fluid communication through the second plurality of perforations 228 at the corners 220. For example, the edges 222 may intersect at substantially a right angle, or about 90 degrees, to define the corners 220. In some embodiments, the corners 220 may have a radius of about 10 millimeters. Further, in some embodiments, three of the second plurality of perforations 228 having a diameter between about 7.75 millimeters to about 8.75 millimeters may be positioned in a triangular configuration at the corners 220 to maximize the exposed surface area for the adhesive 236. In other embodiments, the size and number of the second plurality of perforations 228 in the corners 220 may be adjusted as necessary, depending on the chosen geometry of the corners 220, to maximize the exposed surface area of the adhesive 236. Further, the second plurality of perforations 228 at the corners 220 may be fully housed within the first film layer 204, substantially precluding fluid communication in a lateral direction exterior to the corners 220. The second plurality of perforations 228 at the corners 220 being fully housed within the first film layer 204 may substantially preclude fluid communication of the adhesive 236 exterior to the corners 220 and may provide improved handling of the dressing 110 during deployment at a tissue site. Further, the exterior of the corners 220 being substantially free of the adhesive 236 may increase the flexibility of the corners 220 to enhance comfort.

In some embodiments, the bond strength of the adhesive 236 may vary in different locations of the dressing 110. For example, the adhesive 236 may have a lower bond strength in locations of the first film layer 204 where the second plurality of perforations 228 are relatively larger and may have a higher bond strength where the second plurality of perforations 228 are smaller. The adhesive 236 with lower bond strength in combination with the second plurality of perforations 228 that are larger may provide a bond comparable to the adhesive 236 with higher bond strength in locations having the second plurality of perforations 228 that are smaller, providing a substantially constant overall bond along the periphery 214 of the second surface 212 of the first film layer 204.

The geometry and dimensions of the tissue interface 120, the cover 125, or both may vary to suit a particular application or anatomy. For example, the geometry or dimensions of the tissue interface 120 and the cover 125 may be adapted to provide an effective and reliable seal against challenging anatomical surfaces, such as an elbow or heel, at and around a tissue site. Additionally or alternatively, the dimensions may be modified to increase the surface area for the first film layer 204 to enhance the movement and proliferation of epithelial cells at a tissue site and reduce the likelihood of granulation tissue in-growth.

Further, the dressing 110 may permit re-application or re-positioning to reduce or eliminate leaks, which can be caused by creases and other discontinuities in the dressing 110 and a tissue site. The ability to rectify leaks may increase the reliability of the therapy and reduce power consumption in some embodiments.

Thus, the dressing 110 in the example of FIG. 2 can provide a sealed therapeutic environment proximate to a tissue site, substantially isolated from the external environment, and the negative-pressure source 105 can reduce the pressure in the sealed therapeutic environment. The first film layer 204 may provide an effective and reliable seal against challenging anatomical surfaces, such as an elbow or heel, at and around a tissue site. Further, the dressing 110 may permit re-application or re-positioning, to correct air leaks caused by creases and other discontinuities in the dressing 110, for example. The ability to rectify leaks may increase the efficacy of the therapy and reduce power consumption in some embodiments.

If not already configured, the dressing interface 258 may disposed over the aperture 262 and attached to the cover 125. The fluid conductor 256 may be fluidly coupled to the dressing interface 258 and to the negative-pressure source 105.

Negative pressure applied through the tissue interface 120 can create a negative pressure differential across the first plurality of perforations 226 in the first film layer 204, which can open or expand the first plurality of perforations 226 from their resting state. For example, in some embodiments in which the first plurality of perforations 226 may comprise substantially closed fenestrations through the first film layer 204, a pressure gradient across the fenestrations can strain the adjacent material of the first film layer 204 and increase the dimensions of the fenestrations to allow liquid movement through them, similar to the operation of a duckbill valve. Opening the first plurality of perforations 226 can allow exudate and other liquid movement through the first plurality of perforations 226 into the manifold 202 and the container 115. Changes in pressure can also cause the manifold to expand and contract, and the border 218 may protect the epidermis from irritation. The first film layer 204 can also substantially reduce or prevent exposure of tissue to the manifold 202, which can inhibit growth of tissue into the manifold 202.

In some embodiments, the manifold 202 may be hydrophobic to minimize retention or storage of liquid in the dressing 110. In other embodiments, the manifold 202 may be hydrophilic. In an example in which the manifold 202 may be hydrophilic, the manifold 202 may also wick fluid away from a tissue site, while continuing to distribute negative pressure to the tissue site. The wicking properties of the manifold 202 may draw fluid away from a tissue site by capillary flow or other wicking mechanisms, for example. An example of the manifold 202 being hydrophilic is a polyvinyl alcohol, open-cell foam such as V.A.C. WHITEFOAM™ dressing available from KCI of San Antonio, Texas. Other hydrophilic foams may include those made from polyether. Other foams that may exhibit hydrophilic characteristics include hydrophobic foams that have been treated or coated to provide hydrophilicity.

If the negative-pressure source 105 is removed or turned-off, the pressure differential across the first plurality of perforations 226 can dissipate, allowing the first plurality of perforations 226 to move to their resting state and prevent or reduce the rate at which exudate or other liquid from returning to the tissue site through the first film layer 204.

Additionally or alternatively, instillation solution or other fluid may be distributed to the dressing 110, which can increase the pressure in the tissue interface 120. The increased pressure in the tissue interface 120 can create a positive pressure differential across the first plurality of perforations 226 in the first film layer 204, which can open or expand the first plurality of perforations 226 from their resting state to allow the instillation solution or other fluid to be distributed to the tissue site.

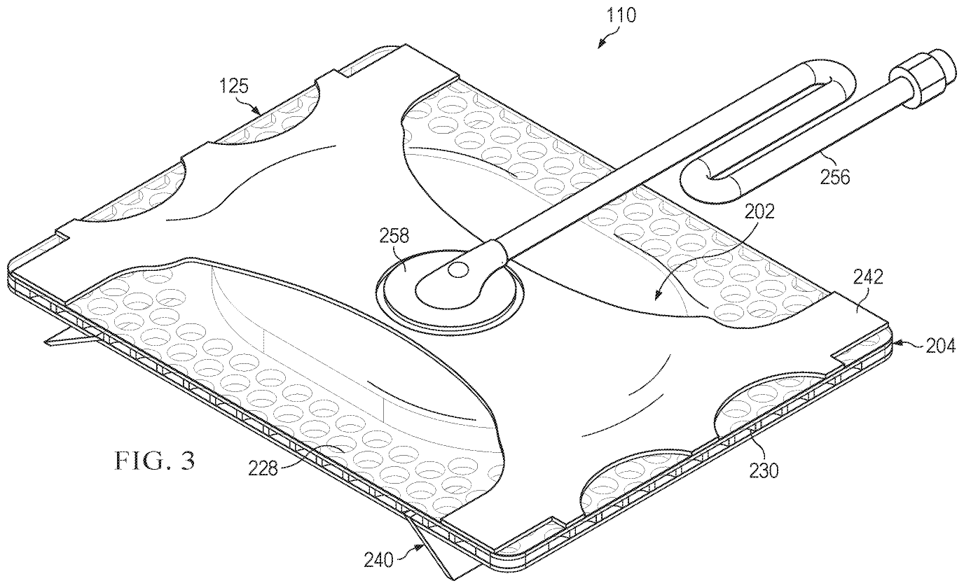

FIG. 3 is an assembly view of the dressing 110 of FIG. 2. When the dressing 110 is assembled, the second surface 234 of the cover 125 may be coupled to the first surface 206 of the manifold 202, the second surface 208 of the manifold 202 may be coupled to the first surface 210 of the first film layer 204, and the second surface 212 of the first film layer 204 may be coupled to the release liner 240. The release liner 240 may be removed from the second surface 212 of the first film layer 204 prior to the dressing 110 being placed at a tissue site. In some embodiments, the carrier 242 may be removed from the dressing 110 after the dressing is placed on a tissue site and prior to the dressing interface 258 being attached to the first surface 232 of the cover 125 of the dressing 110.

Individual components of the dressing 110 may be bonded or otherwise secured to one another with a solvent or non-solvent adhesive, or with thermal welding, for example, without adversely affecting fluid management. Further, the manifold 202 may be coupled to the border 218 of the first film layer 204 in any suitable manner, such as with a weld or an adhesive, for example.

The cover 125, the manifold 202, the first film layer 204, or various combinations may be assembled before application or in situ. For example, the cover 125 may be laminated to the manifold 202 in some embodiments. The first film layer 204 may also be coupled to the manifold 202 opposite the cover 125 in some embodiments. In some embodiments, the dressing 110 may be provided as a single, composite dressing. For example, the first film layer 204 may be coupled to the cover 125 to enclose the manifold 202, with the first film layer 204 being configured to face a tissue site.

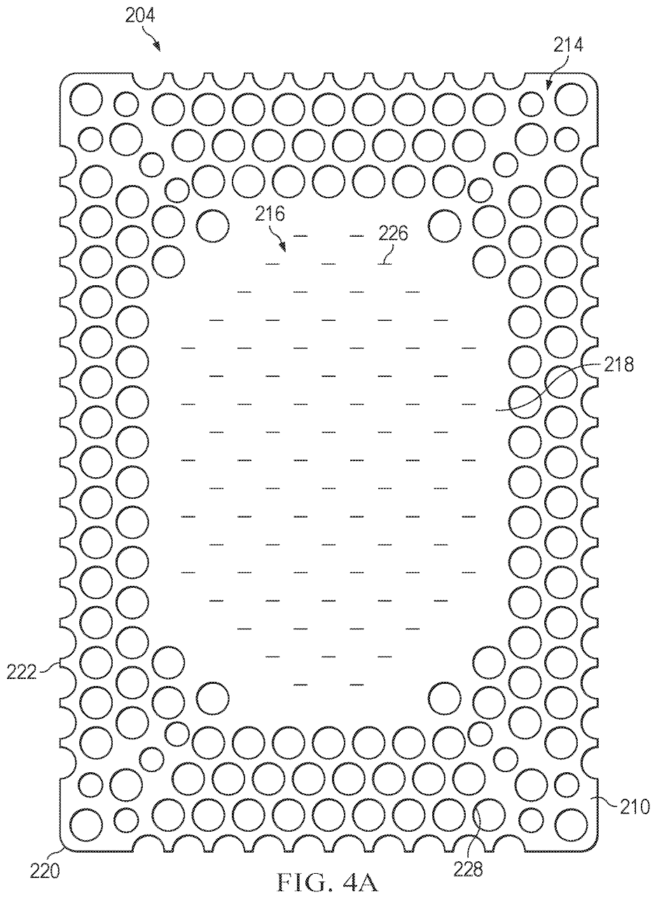

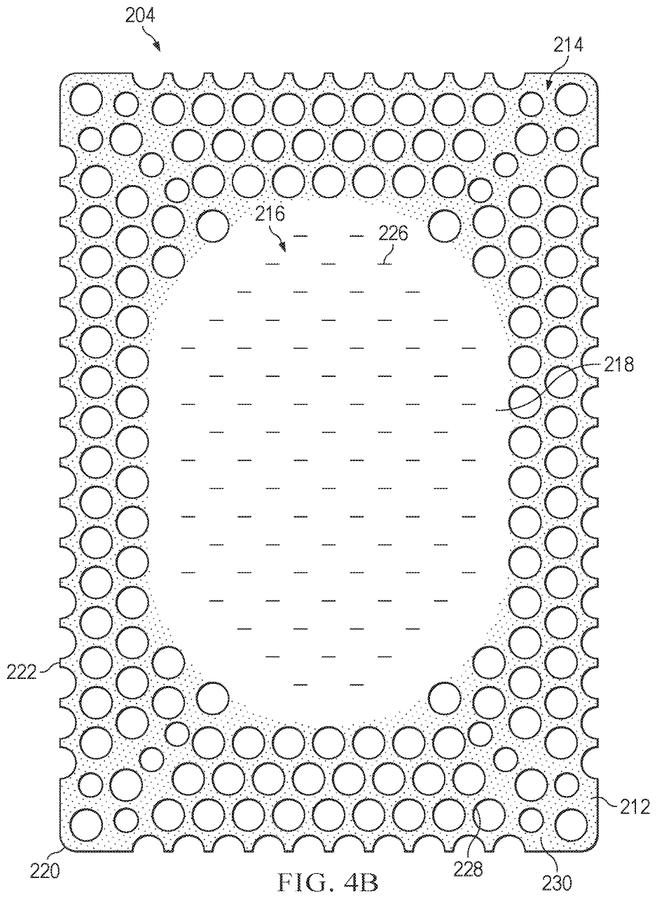

Referring to FIGS. 4A and 4B, the first film layer 204 of the tissue interface 120 is shown. FIG. 4A is a top view of the first film layer 204 and FIG. 4B is a bottom view of the first film layer 204. The first film layer 204 may include the periphery 214, the interior portion 216, and the border 218 that may surround the interior portion 216 and may divide or separate the interior portion 216 from the periphery 214. The sealing adhesive 230 may be disposed on the second surface 212 of the first film layer 204. The sealing adhesive 230 may be disposed on the periphery 214 and may not be disposed on the interior portion 216 of the first film layer 204. The sealing adhesive 230 may be applied to the first film layer 204 such that the interior portion 216 is free from the sealing adhesive 230 and the sealing adhesive 230 surrounds each of the second plurality of perforations 228. In some embodiments, the sealing adhesive 230 may be applied in a pattern to the first film layer 204. Further, in some embodiments, the sealing adhesive 230 may be applied to the periphery 214 of the second surface 212 of the first film layer 204 prior to the first plurality of perforations 226 and/or the second plurality of perforations 228 being formed through the first film layer 204. In some example embodiments, the sealing adhesive 230 may be a layer that may have a portion cut out that corresponds to the interior portion 216 of the first film layer 204. The sealing adhesive 230 may then be laminated to the second surface 212 of the first film layer 204 and the first plurality of perforations 226 can be formed through the interior portion 216 of the first film layer 204 and the second plurality of perforations 228 can be formed through the sealing adhesive 230 and the periphery 214 of the first film layer 204. The first plurality of perforations 226 and the second plurality of perforations 228 may be formed by die-cutting or another method of forming perforations. In some embodiments, the border 218 may also be free from the sealing adhesive 230.

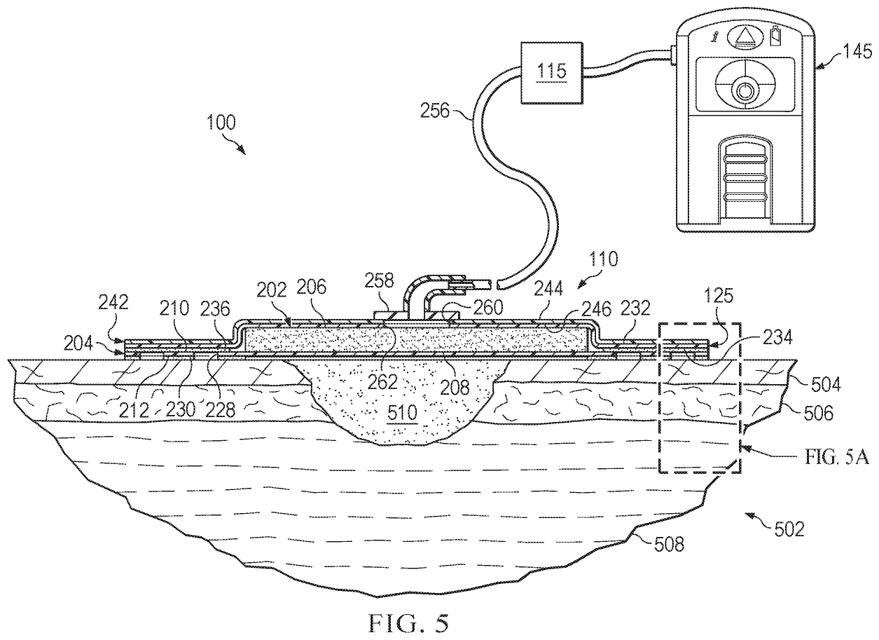

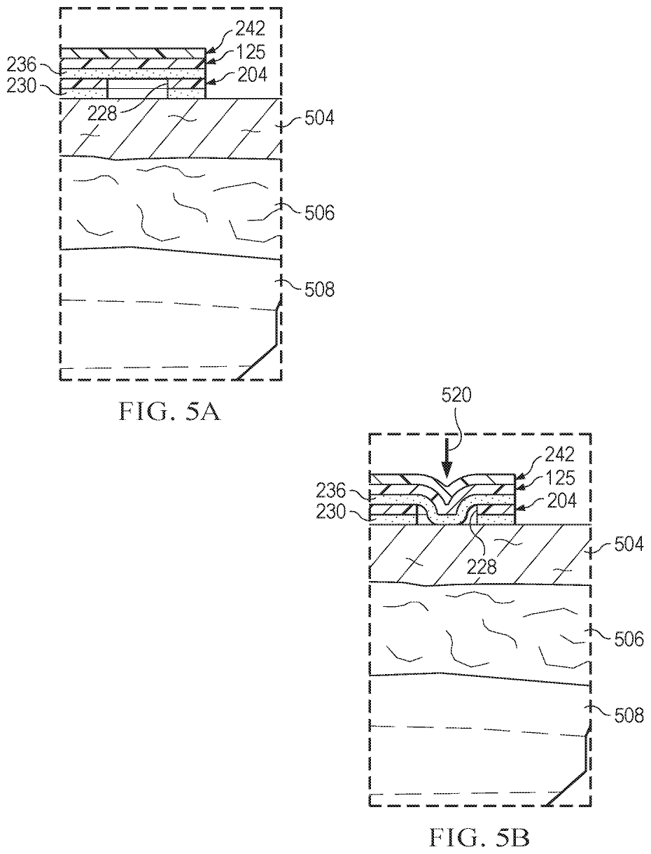

Referring to FIG. 5, the therapy system 100 is shown at a tissue site 502. The tissue site 502 may extend through or otherwise involve an epidermis 504, a dermis 506, and a subcutaneous tissue 508. The tissue site 502 may be a sub-surface tissue site as depicted in FIG. 5 that extends below the surface of the epidermis 504. Further, the tissue site 502 may be a surface tissue site (not shown) that predominantly resides on the surface of the epidermis 504, such as, for example, an incision. The therapy system 100 may provide therapy to, for example, the epidermis 504, the dermis 506, and the subcutaneous tissue 508, regardless of the positioning of the therapy system 100 or the type of tissue site. The therapy system 100 may also be utilized without limitation at other tissue sites.

The therapy system 100 may include the dressing 110, the container 115, and the therapy unit 145 that may include the negative-pressure source 105. Further, the therapy system 100 may include a filler material 510 as an optional component of the therapy system 100 that may be omitted for different types of tissue sites or different types of therapy using negative pressure, such as, for example, epithelialization. If equipped, the filler material 510 may be adapted to be positioned proximate to or adjacent to the tissue site 502, such as, for example, by cutting or otherwise shaping the filler material 510 in any suitable manner to fit the tissue site 502 and to fill a space between the tissue site 502 and the dressing 110. Similar to the manifold 202 of the tissue interface 120, the filler material 510 may be constructed of the manifold materials described herein and may be adapted to be positioned in fluid communication with the tissue site 502 to distribute negative pressure to the tissue site 502. In some embodiments, the filler material 510 may be positioned in direct contact with the tissue site 502 and between the tissue site 502 and the dressing 110. If the filler material 510 is omitted, the tissue interface 120 of the dressing 110 may be positioned in direct contact with the tissue site 502. The dressing 110 may be adapted to provide or distribute negative pressure from the negative-pressure source 105 of the therapy unit 145 to the tissue site 502 directly or through the filler material 510, if equipped.

Referring to FIGS. 5A and 5B, a detail view of the portion of the therapy system 100 of FIG. 5 at reference 5A is shown. FIG. 5A shows the dressing 110 prior to a force being applied to the dressing 110 to push the adhesive 236 through the second plurality of perforations 228 of the first film layer 204. FIG. 5B shows the dressing 110 after a force 520 has been applied to the dressing 110. As discussed above, the adhesive 236 may be in fluid communication with, or otherwise exposed to, an attachment surface through the second plurality of perforations 228 in at least the periphery 214 of the first film layer 204 in some embodiments. In some embodiments, the attachment surface may be the epidermis 504 of the tissue site 502. The adhesive 236 may also be in fluid communication with the edges 222 through the second plurality of perforations 228 exposed at the edges 222. Once the dressing 110 is in the desired position as shown in FIG. 5A, the adhesive 236 may be pressed through the second plurality of perforations 228 to bond the dressing 110 to the epidermis 504. The second plurality of perforations 228 at the edges 222 may permit the adhesive 236 to flow around the edges 222 for enhancing the adhesion of the edges 222 to an attachment surface.

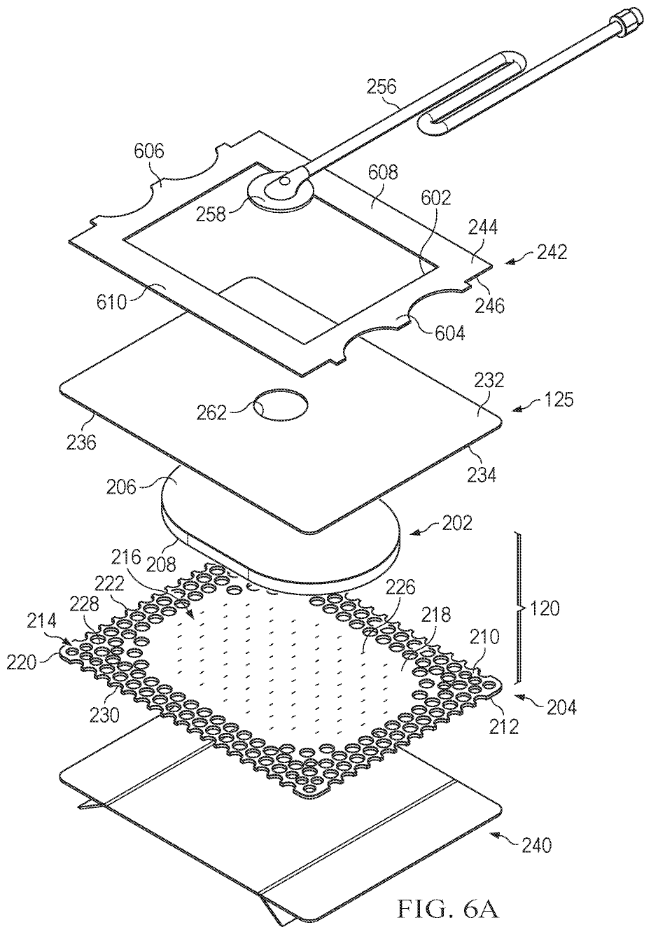

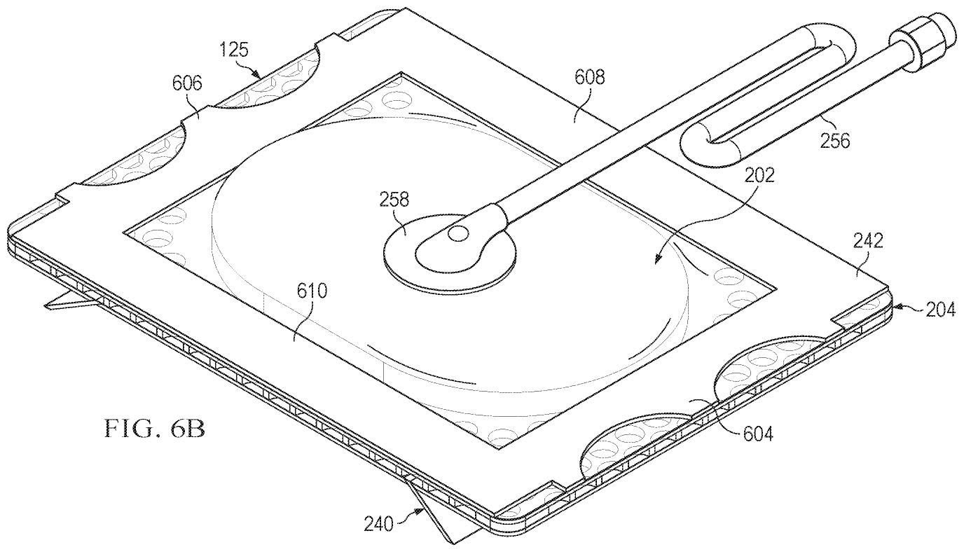

Referring to FIGS. 6A and 6B, another embodiment of the dressing 110 is shown. The cover 125, the tissue interface 120, the dressing interface 258, and the fluid conductor 256 may be substantially as described above with reference to FIG. 2. The carrier 242 may be shaped differently than as shown in FIG. 2 in some embodiments. For example, the carrier 242 may be substantially rectangular and may include a central aperture 602. The carrier 242 may have a first portion 604, a second portion 606 opposite the first portion 604, a third portion 608 coupling the first portion 604 and the second portion 606, and a fourth portion 610 opposite the third portion 608. The dressing interface 258 may couple to the aperture 262 of the cover 125 through the central aperture 602 of the carrier 242. In other embodiments, the carrier 242 may be another shape or size that may provide structure to the dressing 110 before and after the release liner 240 is removed from the dressing 110.

The systems, apparatuses, and methods described herein may provide significant advantages over prior dressings. The dressing 110 may be thin and may be highly conformable because the tissue interface 120 may include the manifold 202 and the first film layer 204 with the sealing adhesive 230 pattern coated to the second surface 212 of the first film layer 204. Because of the thin construction, the dressing 110 may also be breathable. The dressing 110 may also be easier to lift from the tissue site 502 because of the thin edge thickness of the dressing 110.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale.

The appended claims set forth novel and inventive aspects of the subject matter described above, but the claims may also encompass additional subject matter not specifically recited in detail.

For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing configured to be positioned adjacent to a tissue site, comprising:
a first film layer comprising a treatment region and a sealing region around the treatment region, a first plurality of perforations formed through the treatment region, a second plurality of perforations formed through the sealing region that are larger than the first plurality of perforations, and a sealing adhesive covering the sealing region on a first side of the first film layer configured to face the tissue site;
a manifold disposed adjacent a second side of the first film layer, the second side opposite the first side; and
a cover layer comprising a second film layer and a cover adhesive, the cover layer disposed over the manifold and coupled to the second side of the first film layer around the manifold, the cover adhesive disposed adjacent to the second plurality of perforations.

2. The dressing of claim 1, wherein the first film layer comprises a polyurethane film having a thickness of between about 20 microns and about 35 microns.

3. The dressing of claim 1, wherein the sealing adhesive comprises a silicone adhesive having a coating weight of between about 100 grams per square meter and about 250 grams per square meter.

4. The dressing of claim 1, wherein the manifold comprises an open-cell foam having a thickness of between about 5 millimeters and about 10 millimeters.

5. The dressing of claim 1, wherein the second film layer comprises a polyurethane film having a thickness of between about 20 microns and about 35 microns.

6. The dressing of claim 1, wherein the cover adhesive comprises an acrylic adhesive having a coating weight of between about 25 grams per square meter and about 65 grams per square meter.

7. The dressing of claim 1, wherein the cover adhesive is configured to extend through the second plurality of perforations from the second side of the first film layer to the first side of the first film layer.

8. The dressing of claim 1, wherein the treatment region is configured to contact the tissue site and the sealing region is configured to contact epidermis around the tissue site.

9. The dressing of claim 1, wherein the treatment region is free of adhesive such that the first side of the first film layer in the treatment region is configured to be positioned in direct contact with the tissue site.

10. The dressing of claim 1, wherein the first plurality of perforations comprises slits arranged in a pattern.

11. The dressing of claim 1, wherein the second plurality of perforations comprises circular openings arranged in a pattern.

12. The dressing of claim 1, wherein each of the first plurality of perforations comprises a first open area and each of the second plurality of perforations comprises a second open area that is larger than the first open area.

13. The dressing of claim 1, wherein the first plurality of perforations comprise a different shape than the second plurality of perforations.

14. The dressing of claim 1, wherein the second plurality of perforations are larger than the first plurality of perforations in at least one dimension.

15. The dressing of claim 1, wherein the first plurality of perforations are configured to increase in size when the tissue site is exposed to a pressure, wherein the second plurality of perforations do not change in size when exposed to the pressure, and wherein the second plurality of perforations are larger in size than the first plurality of perforations when the tissue site is exposed to the pressure and when the tissue site is not exposed to the pressure.

16. The dressing of claim 1, further comprising a carrier layer releasably coupled to a side of the cover layer opposite the cover adhesive.

17. The dressing of claim 16, wherein the carrier layer comprises a coated paper or a polymeric film.

18. A system for treating a tissue site with negative-pressure therapy, comprising:
the dressing according to claim 1; and
a negative-pressure source configured to be fluidly coupled to the tissue site through the cover layer.

19. A system for treating a tissue site with negative-pressure therapy, comprising:
a dressing configured to be positioned adjacent to the tissue site, comprising:
a carrier layer,
a first film layer comprising a treatment region and a sealing region around the treatment region, a plurality of slits formed through the treatment region, a plurality of holes formed through the sealing region and including a larger open area than the plurality of slits, and a sealing adhesive covering the sealing region on a side of the first film layer configured to face the tissue site,
a manifold, and
a second film layer comprising an aperture, the second film layer coated with a pressure-sensitive adhesive on a side of the second film layer configured to face the tissue site;
wherein the carrier layer, the first film layer, the manifold, and the second film layer are assembled in a stacked relationship with the first film layer and the second film layer enclosing the manifold, the manifold is aligned with the treatment region, the pressure-sensitive adhesive and the sealing adhesive are configured to face the tissue site, at least some of the pressure-sensitive adhesive is exposed through the plurality of holes, and the carrier layer is laminated to the second film layer on a side of the second film layer opposite the pressure-sensitive adhesive; and
wherein a negative-pressure source is configured to be fluidly coupled to the tissue site through the aperture, the manifold, and the plurality of slits.

20. The system of claim 19, wherein the sealing adhesive is not disposed on the treatment region.

21.-27. (canceled)
